(19) United States
(12) Patent Application Publication
(10) Pub. No.: US 2022/0071900 A1
(43) Pub. Date: Mar. 10, 2022

(54) SYSTEMS AND METHODS FOR PREPARING AND TRANSPORTING AN INJECTABLE SLURRY

(71) Applicant: Brixton Biosciences, Inc., Cambridge, MA (US)

(72) Inventors: Sameer SABIR, Arlington, MA (US); Olivier KAGAN, Belmont, MA (US); Charles SIDOTI, Boston, MA (US)

(21) Appl. No.: 17/062,955

(22) Filed: Oct. 5, 2020

Related U.S. Application Data
(60) Provisional application No. 63/075,460, filed on Sep. 8, 2020.

(51) Int. Cl. A61K 9/06; A61K 9/00; A61K 31/047; A61J 1/05

(52) U.S. Cl. CPC A61K 9/06; A61K 9/0019; A61J 2200/44; A61J 1/05; A61K 31/047

(57) ABSTRACT

The present invention provides for a method of transporting and preparing an injectable ice slurry for administration to a patient at a point of care comprising preparing a biocompatible solution comprising water and at least one component other than water, placing the biocompatible solution into a container, transporting the container with the biocompatible solution to the point of care, transforming the biocompatible solution into an injectable ice slurry at the point of care, and administering the injectable ice slurry to the patient at the point of care.

### CROSS-REFERENCE TO RELATED APPLICATIONS

This application claims the benefit under 35 U.S.C. § 119(e), of provisional application U.S. Ser. No. 63/075,460, filed Sep. 8, 2020, entitled “Systems and Methods for Preparing and Transporting an Injectable Slurry,” the entire subjects of which are incorporated herein by reference.

### TECHNICAL FIELD

The present invention relates generally to systems and methods for manufacturing, transporting, storing, and preparing biomaterials. More particularly, the invention relates to manufacturing, transporting, storing, and preparing an injectable slurry for treating a patient at a clinical point of care.

### BACKGROUND

Cold slurries (e.g., ice slurries) are known in the art as compositions that are made of sterile water that forms a plurality of ice particles, as well as excipients, additives, or other components such as freezing point depressants in various amounts, and, optionally, one or more active pharmaceutical ingredients. Such slurries are described in U.S. application Ser. No. 15/505,042 (“'042 application”; Publication No. US2017/0274011), incorporated in its entirety herein. Cold slurries can be administered, preferably via injection, to a tissue of a subject, preferably a human patient, to cause selective or non-selective cryotherapy and/or cryolysis for prophylactic, therapeutic, or aesthetic purposes. Injectable cold slurries may also induce cryoneurolysis and be used to treat various disorders that require inhibition of nerve conduction. For example, U.S. application Ser. No. 15/505,039 (“'039 application”; Publication No. US2017/0274078), incorporated in its entirety herein, discloses the use of slurries to induce reversable degeneration of nerves (e.g., through Wallerian degeneration) by causing crystallization of lipids in the myelin sheath of nerves. The '039 application discloses that this technique can treat various disorders that require inhibition of somatic or autonomic nerves, including motor spasms, hypertension, hyperhidrosis, and urinary incontinence.

A method of preparing a cold slurry is shown in U.S. application Ser. No. 16/080,092 (“'092 application”; Publication No. US2019/0053939). However, the '092 application requires installation of a medical ice slurry production system at the point of care (e.g., near the patient). This technique requires the point of care take steps to maintain sterility of the cold slurry during manufacture and prior to administration.

A method of transporting biological materials is disclosed in U.S. application Ser. No. 15/580,980 which includes transporting a biological material such as blood plasma in a frozen state and thawing the material at the point of care before use. Further, U.S. Pat. No. 10,208,280 discloses a system for storing and transporting biomaterials that includes heat transfer plates for controlling freezing and thawing during transport. Thus, the prior art has been focused on tightly controlling temperature during transport to preserve the stability or therapeutic utility of the biomaterial. These disclosures do not address the issue of easily forming a therapeutic biomaterial, such as an injectable cold slurry, at the point of care, and instead require complex and expensive transport vessels for regulating the temperature of the biomaterial during storage and transport.

Therefore, there exists a need of easily transporting a sterile biomaterial to a point of care using standard shipping techniques, where the temperature of the biomaterial is not controlled during transport and the sterility of the biomaterial is maintained during transport, and the biomaterial can be transformed into a therapeutic state, such as a flowable and injectable cold slurry, at a point of care without requiring manufacturing equipment to be available at the point of care and without compromising the sterility of the biomaterial at the point of care. The present disclosure addresses this need by providing for improved apparatuses, systems, and methods of transporting, storing, and preparing a biomaterial, such as an injectable slurry, for administration to a patient or subject at a point of care. The present disclosure provides a simpler method of transport that better preserves the sterility of the biomaterial and reduces the time required to provide a therapeutic substance to a patient.

### SUMMARY

In one aspect, the invention provides for a method of transporting and preparing an injectable ice slurry for administration to a patient at a point of care comprising: preparing a biocompatible solution comprising water and at least one component other than water, placing the biocompatible solution into a container, transporting the container with the biocompatible solution to the point of care, transforming the biocompatible solution into an injectable ice slurry at the point of care, and administering the injectable ice slurry to the patient at the point of care.

In some embodiments, the at least one component other than water is glycerol or a derivative thereof. In some embodiments, the at least one component other than water is a salt or a derivative thereof. In some embodiments, the salt is sodium chloride. In some embodiments, the biocompatible solution further comprises glycerol or a derivative thereof, and sodium chloride or a derivative thereof. In some embodiments, an amount of glycerol or a derivative thereof in the biocompatible solution is selected from the group consisted of about 30% (v/v) of the biocompatible solution, about 20% (v/v) of the biocompatible solution, and about 10% (v/v) of the solution. In some embodiments, the water constitutes about 80% (w/v) of the biocompatible solution. In some embodiments, the biocompatible solution is configured to be transported to and stored at the point of care in a non-temperature-controlled environment prior to transforming the biocompatible solution into the injectable ice slurry. In some embodiments, the transforming includes modifying the biocompatible solution, wherein the modifying is selected from the group consisting of mechanical agitation, blending, mixing, vibration, ultrasonic energy, manual shaking, freezing, thawing, and a combination thereof. In some embodiments, the container is transported to the point of care in a support vessel and wherein the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of frozen ice particles. In some embodiments, the biocompatible solution is configured to be transformed into the injectable ice slurry by placing the container into a freezer at the point of care. In some embodiments, the biocompatible solution comprises a plurality of ice particles and is configured to flow through a lumen used for administration of the injectable ice slurry to the patient.

In another aspect, the invention provides for a method of transporting a container that has been pre-filled with a biocompatible solution to a point of care comprising: preparing the biocompatible solution comprising water and at least one component other than water, placing the biocompatible solution into a container, and transporting the container with the biocompatible solution to the point of care in a support vessel, wherein the biocompatible solution is transformed into an injectable ice slurry at the point of care, and wherein the injectable ice slurry is configured to be administered to a patient at the point of care.

In some embodiments, the at least one component other than water is glycerol or a derivative thereof. In some embodiments, the biocompatible solution comprises water and glycerol or a derivative thereof, and sodium chloride or a derivative thereof. In some embodiments, the biocompatible solution is configured to be transformed into the injectable ice slurry by placing the container into a freezer at the point of care. In some embodiments, the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of frozen ice particles. In some embodiments, the injectable slurry comprises 10% ice by weight, between about 10% ice by weight and about 20% ice by weight, between about 20% ice by weight and about 30% ice by weight, between about 30% ice by weight and about 40% ice by weight, between about 40% ice by weight and about 60% ice by weight, or more than about 60% ice by weight.

In another aspect, the invention provides for a method of administering an injectable ice slurry at a point of care to a patient comprising: receiving at a point of care a container comprising a biocompatible solution, transforming the biocompatible solution into the injectable ice slurry, administering the injectable ice slurry to the patient, and wherein the biocompatible solution comprises water and at least one component other than water.

In some embodiments, the biocompatible solution further comprises glycerol or a derivative thereof, and sodium chloride or a derivative thereof. In some embodiments, the amount of glycerol or a derivative thereof in the biocompatible solution is selected from the group consisted of about 30% (v/v) of the biocompatible solution, about 20% (v/v) of the biocompatible solution, and about 10% (v/v) of the biocompatible solution. In some embodiments, the container is received in a sealed state configured to maintain sterility of the biocompatible solution and of the injectable ice slurry. prior to the administration of the injectable ice slurry to the patient. In some embodiments, the container is placed in a freezer prior to transforming the biocompatible solution into the injectable ice slurry. In some embodiments, the container with the biocompatible solution is transported to the point of care in a support vessel, and wherein the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of frozen ice particles.

In another aspect, the invention provides for a container comprising: a sterile biomaterial comprising water and at least one component other than water, wherein the container is configured to be transported to a point of care without breaking a sterile barrier of the container, wherein the sterile biomaterial is transformed into an injectable slurry at the point of care while inside the container, and wherein the injectable slurry is administered to a patient at the point of care.

In some embodiments, the sterile biomaterial further comprises glycerol or a derivative thereof, and sodium chloride or a derivative thereof. In some embodiments, the amount of glycerol or a derivative thereof in the biocompatible solution is selected from the group consisted of about 30% (v/v) of the biocompatible solution, about 20% (v/v) of the biocompatible solution, and about 10% (v/v) of the biocompatible solution. In some embodiments, transforming the sterile biomaterial into the injectable slurry includes modifying a contents of the container, wherein the modifying is selected from the group consisting of mechanical agitation, blending, mixing, vibration, ultrasonic energy, manual shaking, freezing, thawing, and a combination thereof. In some embodiments, the sterile barrier of the container is configured to be maintained until the injectable slurry is administered to the patient. In some embodiments, the sterile biomaterial is configured to be transformed into the injectable ice slurry by placing the container into a freezer at the point of care.

### BRIEF DESCRIPTION OF THE DRAWINGS

The following figures depict illustrative embodiments of the invention.

FIG. 1 depicts a freezing point depression graph for water, a solution containing 10% glycerin volume by volume (v/v), and a solution containing 20% glycerin (v/v).

FIG. 2 is a table showing the breakdown by volume and weight of components of an exemplary biomaterial that can form an injectable slurry.

FIG. 3 is a graph of solid to liquid phase transitions of cold slurries having crystallization set points of −5.5° C. and −8.1° C.

FIG. 4 is a diagram depicting a method of preparation, transport, storage, and delivery of a slurry to a patient or subject at a point of care.

### DETAILED DESCRIPTION

The present disclosure is directed to a method of transporting to a point of care a sterile biomaterial or solution in a container. The term biomaterial and solution are used interchangeably throughout this disclosure. The biomaterial is preferably not temperature-controlled during transport and can be transformed or manipulated at the point of care, prior to administration to a subject or patient, without breaching the sterile barrier of the container. Preferably, the container with the biomaterial is transporting using standard shipping methods, e.g., U.S.P.S., FedEx, or UPS. Further, in a preferred embodiment, the sterile biomaterial can be transformed into a therapeutic substance at the point of care using standard equipment or appliances available at the point of care or using a component in the container and/or shipping vessel (e.g., a box holding container during shipment). The sterile barrier of the container is not compromised when the biomaterial is transformed. In a preferred embodiment, the biomaterial is transformed into a flowable and injectable slurry (e.g., a mixture of solid ice particles suspended in a liquid solution) at the point of care using a standard freezer and, optionally, a component(s) provided in the shipping vessel and/or in the container (e.g., a component within the syringe holding the biomaterial). The container may also optionally be subjected to thawing at the point of care prior to administration to a subject or patient.

In some embodiments, the biomaterial is a cold slurry (e.g., ice slurry) that can be delivered via injection directly to a tissue of a human patient or a subject for prophylactic, therapeutic, or aesthetic purposes as disclosed in the '042 application. The injectable slurry can be used for selective or non-selective cryotherapy, cryolysis, or cryoneurolysis. In some embodiments, the therapeutically effective injectable slurry is comprised entirely of water and non-active excipient or additional component materials. In other embodiments, the slurry further comprises a known active pharmaceutical compound.

In some embodiments, the sterile biomaterial (or solution) is placed into a sterile container, such as a syringe, a vial, a bag, or a plastic or glass vessel, prior to the container being shipped to the point of care. Preferably, the sterile biomaterial is made of water, at least one salt, such as sodium chloride, and at least one additional excipient or component (other than salt), such as glycerol, with each component making up a certain percentage of the total volume of the biomaterial. The container is then shipped to the point of care without the need for temperature control during transport. The biomaterial within the container is preferably in its aqueous state at about room temperature during transport. In some embodiments, the stability and sterility of the biomaterial is maintained throughout transport. In other embodiments, the biomaterial is sterilized at the point of care. The biomaterial may also be stored without temperature control (e.g., at about room temperature) at the point of care or at an intermediate storage facility before arrival at the point of care. At the point of care, the biomaterial is conditioned or transformed to form a flowable and, preferably, injectable cold slurry while remaining inside the container. This conditioning or transformation is preferably performed without breaking the sterile boundary of the container and without compromising the sterility of the biomaterial. This conditioning or transformation can include one or more of: placing the container into a standard freezer set at a predetermined temperature; physically manipulating the contents of the container such as through mechanical agitation; or subjecting the internal contents of the container to thawing. Preferably, the conditioning or transformation step(s) results in an injectable cold slurry with a predetermined percentage of ice particles. In some embodiments, the injectable slurry will have ice particles of predetermined size(s). The cold slurry is then administered to a patient or subject, preferably by injection. The individual administering the cold slurry can then dispose of the container using conventional means. In some embodiments, the point of care does not need to install any additional equipment to prepare the injectable cold slurry. In other embodiments, the point of care will use equipment configured to transform the biomaterial into an injectable slurry after the biomaterial arrives at the point of care. In some embodiments, the container can undergo a process to transform its contents into an injectable slurry using only components that are shipped to the point of care with the container, such as components of the packaging, or components included within the container, as well as standard equipment available at the point of care, such as a freezer. In other embodiments, the point of care can purchase separate equipment that can be used with the shipped biomaterial (and/or its packaging) to transform the biomaterial into an injectable slurry.

In some embodiments, the final product to be administered via injection to a human patient or a subject (such as a human who is not a patient or a non-human animal) is a cold slurry comprised of sterile ice particles of water and varying amounts of excipients, additives, or additional components such as freezing point depressants. For example, the percentage of ice particles in the cold slurry can constitute less than about 10% by weight of the slurry, between about 10% by weight and about 20% by weight, between about 20% by weight and about 30% by weight, between about 30% by weight and about 40% by weight, between about 40% by weight and about 60% by weight, more than about 60% by weight, and the like. The sizes of the ice particles will be controlled to allow for flowability through a vessel of various sizes (e.g., needle gauge size of between about 7 and about 43) as described in the '042 application. Further, other methods may be used to condition the size of the ice particles to allow for flowability through a vessel of various sizes. In some embodiments, the majority of ice particles have a diameter that is about half of the internal diameter of the lumen or vessel used for injection. For example, ice particles can be about 1.5 mm or less in diameter for use with a 3 mm catheter. In some embodiments, the distribution of the diameter of ice particles is unimportant, as long as the final product is a flowable and injectable ice slurry and can be injected using a syringe needle of a predetermined size.

There are a variety of techniques that may be used to prepare a solution that can form an ice slurry. This disclosure is not limited to any particular method or technique. In some embodiments, one or more excipients or additional components may be included in the slurry. An excipient is any substance that is not itself a therapeutic agent used as a diluent, adjuvant, and/or vehicle for delivery of a therapeutic agent to a subject or patient, and/or a substance added to a composition to improve its handling, stability, or storage properties. Excipients or additional components can constitute less than about 10% volume by volume (v/v) of the slurry, between about 10% v/v and about 20% v/v of the slurry, between about 20% v/v and about 30% v/v, between about 30% v/v and 40% v/v, and greater than about 40% v/v. Various added excipients or additional components can be used to alter the phase change temperature of the slurry (e.g., reduce the freezing point), alter the ice percentage of the slurry, alter the viscosity of the slurry, prevent agglomeration of the ice particles, prevent dendritic ice formation (i.e., crystals with multi-branching “tree-like” formations, such as those seen in snowflakes), keep ice particles separated, increase thermal conductivity of fluid phase, or improve the overall prophylactic, therapeutic, or aesthetic efficacy of the injectable slurry.

One or more freezing point depressants can be added as excipients or additional components to form slurries with freezing points below 0° C. Depressing the freezing point of the slurry allows it to maintain flowability and remain injectable while still containing an effective percentage of ice particles. Suitable freezing point depressants include salts (e.g., sodium chloride, betadex sulfobutyl ether sodium), ions, Lactated Ringer's solution, sugars (e.g., glucose, sorbitol, mannitol, hetastarch, sucrose, (2-Hydroxypropyl)-β-cyclodextrin, or a combination thereof), biocompatible surfactants such as glycerol (also known as glycerin or glycerine), other polyols (e.g., polyvinyl alcohol, polyethylene glycol 300, polyethylene glycol 400, propylene glycol), other sugar alcohols, or urea, and the like. Other exemplary freezing point depressants are disclosed in the '042 application and are incorporated in their entirety herein.

The concentration of freezing point depressants will determine the ice particle percentage of the slurry and its flowability and injectability. The degree of freezing point depression can be calculated using the following formula as described in the '042 application, incorporated herein:

ΔT_{F}=K_{F}bi

wherein ΔT_{F} is the freezing point depression (as defined by T_{F (pure solvent)}−T_{F (solution)}), K_{F} is the cryoscopic constant, b is molality, and i is the van't Hoff factor representing the number of ion particles per individual molecule of solute. Other methods of computing freezing point depression can also be used, as disclosed in the '042 application.

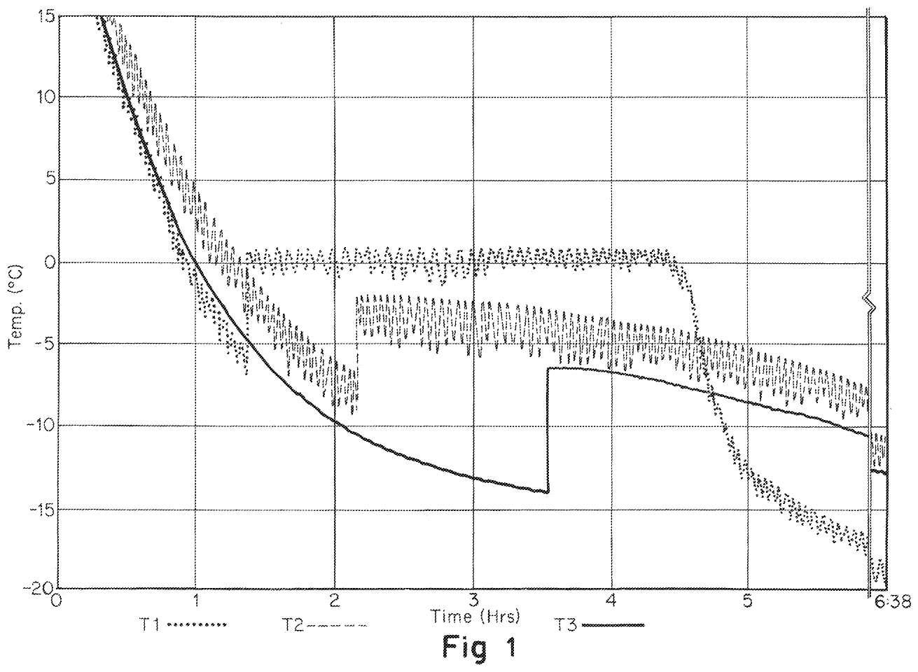

Referring to FIG. 1, a freezing point depression graph is shown for pure water T1, a mixture of water and 10% (v/v) glycerin T2, and a mixture of water and 20% (v/v) glycerin T3. In this graph, all the substances were placed in a freezer having a constant temperature of −20° C. The temperature was measured using a thermometer placed in each substance. The graph shows that a mixture of water and glycerin will have a different freezing point than that of pure water, which means the solution can be cooled to below 0° C. and only be partially crystallized. The graph shows that cooling causes pure water T1 to crystallize at an equilibrium freezing point of 0° C. This is indicated by the period of time where the pure water remains at a temperature of about 0° C., from about 1.3 hours to about 4.4 hours, which begins immediately after pure water T1 passes a supercooling point at about −6° C. Having an equilibrium window of crystallization (i.e., the “flat line” portion of pure water T1 in FIG. 1) is typical for a pure solvent. For the 10% glycerin solution T2, cooling causes the solution to begin crystallizing at an initial freezing point of about −3° C. after about 2.2 hours, and the crystallization continues as the temperature of the solution drops further to about −8° C. after about 6 hours. The initial crystallization occurs immediately after 10% glycerin solution T2 passes a supercooling point at about −8° C. (which can vary from sample to sample, e.g., supercooling point of between about −15° C. and about −3° C.), shown at around 2.2 hours. Having a descending temperature window of crystallization for the 10% glycerin solution T2 is typical for a solution (i.e., impure mixture). Similarly, for the 20% glycerin solution T3, cooling causes the solution to begin crystallizing at an initial freezing point of about −7° C. after about 3.5 hours (following an initial supercooling point which can vary from sample to sample, e.g., between about −25° C. and about −5° C.), and the crystallization continues as the temperature of the solution drops further to about −11° C. after about 6 hours and continues to decline thereafter past 6.5 hours. The initial crystallization occurs immediately after 20% glycerin solution T3 passes a supercooling point at about −14° C., shown at around 3.5 hours. Similar to the trace for 10% glycerin solution T2, the descending temperature window of crystallization for 20% glycerin solution T3 is typical for a solution.

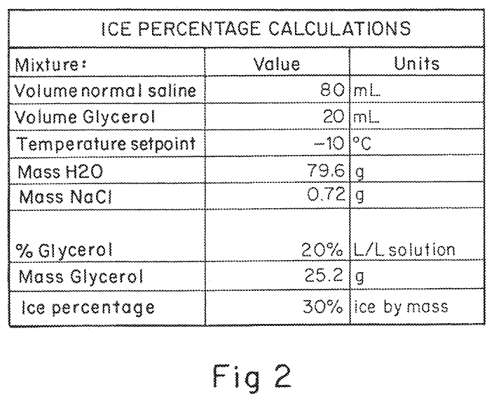

Referring to FIG. 2, this chart shows the components of an exemplary biomaterial that can form a slurry. This chart shows that the percentage of ice for an exemplary biomaterial can be calculated for a particular temperature. The exemplary slurry contains 30% ice by mass (weight by weight; w/w) at −10° C. This exemplary slurry has 80 mL of saline (0.9% NaCl) and 20 mL of glycerol (i.e., glycerin). In weight, such a slurry has about 79.6 g of pure water, about 0.72 g of sodium chloride, and about 25.2 g of glycerol (approximately 20% v/v). In other embodiments, the slurry could contain higher or lower percentages of glycerol by adjusting the relative volume of glycerol to saline. For example, other suitable slurries contain about 10% glycerol (v/v), between about 10% and about 20% glycerol, about 30% glycerol, or more than about 30% glycerol. If an active pharmaceutical compound is to be added to the slurry, the concentration of saline can be adjusted accordingly to maintain the desired concentration of excipients or additional components such as glycerol. The percentage of ice will vary depending on the composition of the biomaterial.

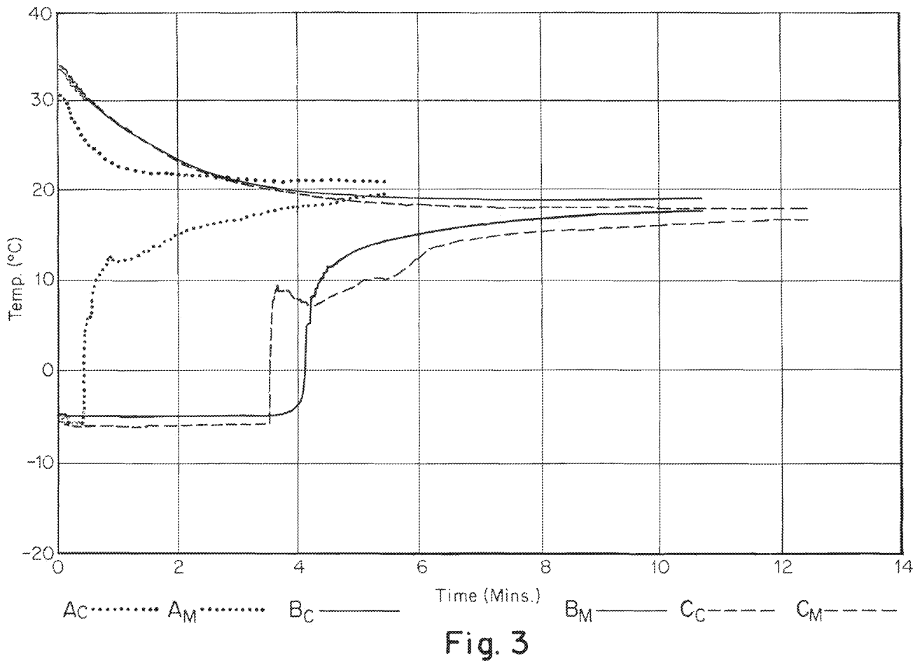

Referring to FIG. 3, different slurry compositions (batches) are characterized with respect to their temperature profiles. The different slurry batches were placed into a copper plate that is heated to 40° C. and has thermocouples placed to measure the change in temperature of the slurry over time. The plotted data shows temperature change over time for three different slurry batches. The temperatures are measured at two different positions for each slurry: embedded inside of the copper plate (traces A_{C}, B_{C}, and C_{C}) and projecting out from the middle of the copper plate into the slurry (traces A_{M}, B_{M}, and C_{M}). The temperature traces show three separately created slurry batches: a slurry composition having 15% glycerin (having a temperature setpoint of −8.1° C.) is represented by traces A_{C} and A_{M}, and two different slurry batches both having 10% glycerin (having a temperature setpoint of −5.5° C.) are represented by traces B_{C} and B_{M}, as well as traces C_{C} and C_{M}. When a slurry batch is first introduced into the copper plate, the thermocouple wire embedded inside the plate (traces A_{C}, B_{C}, and C_{C}) initially measures the warm temperature of the heated plate (e.g., 31° C. for trace A_{C} at timepoint 0) and then reaches an equilibrium at a lower temperature due to the cooling effect of the introduced slurry (e.g., 22° C. for trace A_{C} at around 2 minutes). On the other hand, for the thermocouple wire located in the middle of the plate, when a slurry is first introduced into the copper plate it immediately contacts the thermocouple wire since that wire is exposed. This causes an initially negative temperature reading in the middle position due to the crystallized slurry contacting the wire (e.g., −5° C. for trace A_{M} at timepoint 0) followed by an equilibrium at a warmer temperature as the slurry begins to melt on the heated plate (e.g., 18° C. for trace A_{M} at around 4 minutes). The thermocouple wire exposed to the outside of the plate (traces A_{M}, B_{M}, and C_{M}) can be used to detect phase transitions during which the crystallized slurry begins to melt. The graph shows that the two slurry compositions with 10% glycerin reach their phase transition at similar timepoints (at around 4 minutes for trace B_{M}, and at around 2.7 minutes for trace C_{M}), which differ from the phase transition for the 15% glycerin slurry (phase transition occurs at around 0.2 minutes for trace A_{M}). The graph also shows that the two slurry batches having the same composition (10% glycerin: traces B_{C} and B_{M} and traces C_{C} and C_{M}) reach equilibrium (as measured by the two thermocouple wire positions) in a similar time frame and at similar temperatures of between about 15° C. and 19° C. depending on the location of the thermocouple (middle/bottom). On the other hand, the slurry with a different composition (15% glycerin: traces A_{C} and A_{M}) has a different temperature profile from the other two, reaching an equilibrium sooner at the temperature of between about 19° C. and 22° C. depending on the location of the thermocouple (middle/bottom). FIG. 3 therefore demonstrates that slurries of different compositions have different temperature profiles and batch to batch consistency exists across slurries having the same composition (e.g., the slurry represented by B_{C} and B_{M} and slurry represented by C_{C} and C_{M} have similar temperature profiles which is different from that of slurry represented by A_{C} and A_{M}).

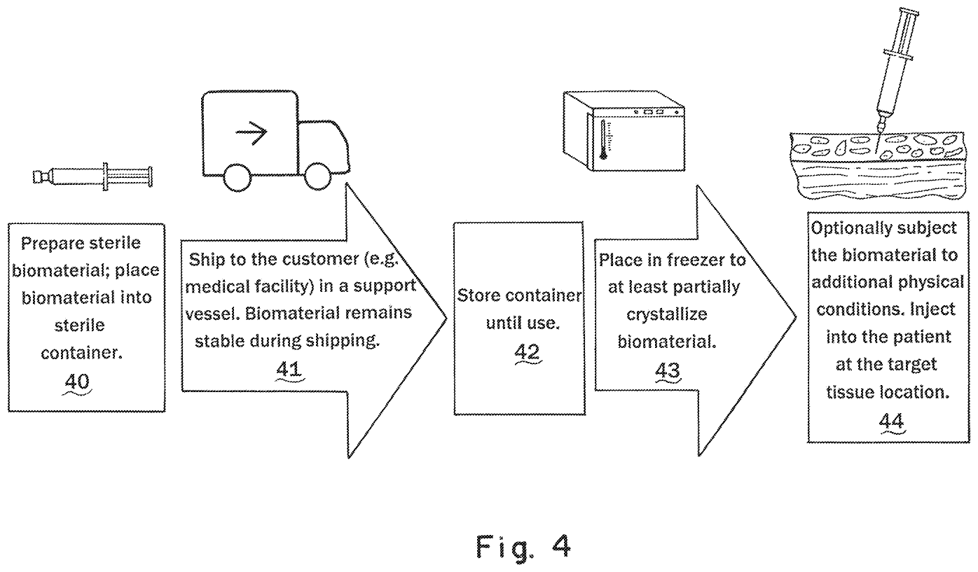

FIG. 4 provides a diagram of one embodiment of the present disclosure. This diagram shows a method of transporting to a point of care a biomaterial to be injected into a subject or patient. At step 40 a biomaterial that will be injected is prepared according to any method provided herein or any method disclosed in the '042 application. In a preferred embodiment, the preparation step includes mixing an amount of pure water with an amount of excipient, additional component, or additive. Preferably, the prepared material is sterile. The solution is prepared for shipment in its aqueous phase (i.e., without any ice content). Alternatively, it is also possible to prepare the solution in its crystallized or slurry form (i.e., with a plurality of ice particles). The solution (or slurry) at step 40 is placed into a container. In some embodiments, the container can be a syringe, a vial, a bag, a plastic or glass vessel, or any other container with a sealed internal volume. Such a container may be made of any material known in the art, e.g., plastic, polystyrene, polyolefin, high density polyethylene, etc. The container shown in FIG. 4 is a syringe. The solution (or slurry) may be sterile and biocompatible when prepared. In such an embodiment, the solution (or slurry) is placed into the container while maintaining the sterility and biocompatibility of the solution and of the internal container environment. In an alternative embodiment, the container is sterilized after the solution (or slurry) is placed within the container.

The container that is filled in step 40 is preferably compatible with warm and cold temperature conditions and is preferably able to withstand ionizing irradiation for sterilization purposes. The container is preferably able to withstand exposure to a variety of temperature environments including ranges from about −80° C. to about +80° C. The container may have an internal volume ranging from about 0.1 mL to about 10 L, for example. Irrespective of the volume capacity of the container, the solution (or slurry) can be introduced into the container at various volumes at or below the volume capacity of the container such as between about 25% and about 50%, at about 50% of the capacity, between about 50% and about 80% of the capacity or at above about 80% of the capacity. Further, the container may comprise one or more conduits to allow filling or draining of the solution (or slurry) into and/or out of the interior of the container. Such conduits preferably maintain the sterility of the contents of the container. At the point of care, such conduits can be accessed to allow transfer of the solution (or slurry) from the container to a syringe or any other device prior to administration to a patient via injection. The container may also include a visible temperature indicator that can allow for visual monitoring of the temperature of the biomaterial, or the approximate temperature of the biomaterial. The temperature indicator can be a temperature sensing label, sticker, marker, crayon, lacquer, pellet, etc., including reversible temperature labels that can dynamically track temperature changes. The temperature indicator can be located inside the container (e.g., a pellet placed directly into the internal solution), on the inside walls of the container, on the outside walls of the container, or in any location that allows for visual tracking of the temperature of the contents inside the container.

The solution may be placed in the container in its aqueous phase or in slurry form. In embodiments in which the container is a syringe, any syringe that is suitable for administering a slurry via injection may be used. Syringes that can hold a variety of volumes may be used, such as 0.5 mL, 1 mL, 2 mL, 5 mL, or greater than 5 mL syringes, and the like. Various syringe tips (i.e., the part of the syringe which forms a connection with a needle) are contemplated for use with the present disclosure such as secure screw, slip/push-on, eccentric, catheter, permanently attached, etc. It is further contemplated that the syringes for use with the present disclosure may have a variety of different needle gauge sizes (e.g., the needle gauge sizes set forth in Table 2 of the '042 application). In some embodiments, the needle gauge size is about 20. The syringes for use with the present disclosure may also have a variety of needle lengths (e.g., smaller than about ⅜ of an inch, between about ⅜ of an inch and about ¾ of an inch, between about ¾ of an inch and about 1 and ¼ inches or greater than about 1 and ¼ inches). The syringe may be made of any suitable medical grade material known in the art such as plastic or glass, including freezer-safe materials. The syringe maintains internal sterility and overall structural stability throughout exposure to a wide range of temperature conditions including temperatures ranging from about −80° C. to about +80° C. The syringe can be packaged for transport alongside a compatible needle or plurality of needles; it may also be packaged without a needle. In such an embodiment, the needle can be provided at the point of care prior to administration to a patient. Alternatively, the needle may be permanently attached to the syringe.

At step 41, the container that has had the solution placed inside of it is transported to the point of care, such as a medical facility (e.g., physician's office, clinic, or hospital). Thus, the point of care receives a prefilled container, e.g., a prefilled syringe. Other points of care can include a patient's residence, a cosmetic services facility/clinic, an investigational laboratory, etc. The prefilled container is prepared for transport by being placed in any suitable support vessel for shipping known in the art. The support vessel is configured to maintain sterility of the container's contents and to protect the container from damage during transport (e.g., rupture, spillage, and/or compromise of internal sterility). The support vessel may be comprised of multiple vessels; for example, a smaller vessel can hold the container which is then placed in a larger vessel that contains transport protections such as bubble wrap or other padding. The solution is stable when transported at room temperature at step 41. Alternatively, the solution may be transported under a variety of temperature ranges at step 41 including from above room temperature, such as about 35° C. to about room temperature, at about 20° C., between about 20° C. and about 0° C., between about 0° C. and −10° C., between about 10° C. and −20° C., or colder than about −20° C. In some embodiments, the solution (or slurry) is transported at a temperature of between about 0° C. and about −20° C. In some embodiments, the solution (or slurry) is maintained at stable sub-0° C. temperatures throughout transport. The support vessel is suitable for maintaining cold temperatures by comprising one or more ice packs, dry ice, or similar items. The vehicle used for transport at step 41 can be any standard shipping vehicle such as a standard motor vehicle, an unmotorized vehicle (e.g., bicycle), a truck, a marine vessel (e.g., ship, boat, etc.), an airplane, or similar vehicles. The transport vehicle may also be an ambulance or fire truck which transports the solution (or slurry) as part of an emergency response. Alternatively, it is also possible for transport step 41 to involve physical delivery of the support vessel by foot. Any combination of such transportation modes can be used in a sequence of steps until the point of care destination is reached.

At step 42, the prefilled container is stored until use at the point of care, or at an intermediate storage facility. In an embodiment, the prefilled container can be removed from the support vessel upon arrival. In an alternative embodiment, the prefilled container is stored in the support vessel. The container can be stored at a variety of temperatures, including above room temperature, at room temperature, or in a freezer at a variety of suitable freezer temperatures, including below about −19° C., between about −19° C. and 2° C., in a refrigerator at refrigeration temperatures of between about 2° C. and about 6° C., or at warmer than about 6° C. The container can be subjected to the above-mentioned storage temperature conditions in its unaltered state upon removal from the support vessel. In some embodiments, a slurry is received at the point of care in at least a partially crystalized form at a temperature of between about 0° C. and about −20° C. The sterility and stability of the biomaterial in the container is maintained during storage.

At step 43, the container is removed from storage and placed in a freezer if the container has not been stored in a freezer. When the container is exposed to freezing temperatures, e.g., between about −25° C. and about −19° C., between about −19° C. and about −10° C. and between about −10° C. and about 0° C., the solution inside the container may be altered from being in an aqueous state to being an injectable slurry having a percentage of ice particles, e.g., about 10% ice by weight, between about 10% ice by weight and about 20% ice by weight, between about 20% ice by weight and about 30% ice by weight, between about 30% ice by weight and about 40% ice by weight, between about 40% ice by weight and about 60% ice by weight, more than about 60% ice by weight, as previously described herein. As described previously, a user will be able to calculate the percentage of ice particles that will form in a given solution at a particular temperature based on the solution's components. For example, if a user were to place a container having the biomaterial described in FIG. 2 into a freezer that had a set temperature of −10° C., the user would know that the solution would have 30% ice particles by mass after reaching −10° C. Alternatively, or in addition to exposure to freezing temperatures, the container may be exposed to conditions of pressure and/or humidity changes.

In some embodiments, the prefilled solution (or slurry) is stored at about room temperature at step 42. The solution can transform into an injectable slurry by being placed in a freezer at step 43. The slurry can then be directly administered to the patient at step 44 after removal from the freezer. In an alternative embodiment, the slurry can be subjected to additional state transformation steps as discussed below.

In an alternative embodiment, a percentage of the aqueous solution turns into solid ice (or partially crystallized) at step 43. In such an embodiment, the particle sizes of the ice may be too large for injection via a needle. In such an embodiment, the container is removed from the freezer at step 44 and subjected to one or more conditioning/state transformation methods that transform the frozen container contents into an injectable and flowable slurry. For example, the container contents can be subjected to mechanical agitation, blending, mixing, vibration, ultrasonic energy, manual shaking, thawing, or any combination thereof. Additionally, the sizes of ice particles in the slurry can be further modulated by processing the slurry through methods such as filtering, screening, or sorting of the ice particles. The container contents are subjected to these conditioning/state transformation methods without breaking the sterile barrier of the container (or syringe). Such mechanical agitation may be implemented by components located inside of the container/syringe, or by components located in the support vessel in which the container was shipped. Further, the container/syringe may operate in conjunction with an external adapter component that is operable to transform the contents into an injectable and flowable slurry. Such an adapter component may be included in the support vessel in which the container was shipped or may already be stored at the point of care or purchased separately.

Alternatively, or in addition, the container can be subjected to one or more of the described state transformation methods prior to storage at step 42. The temperature of the freezer at step 43 may be controlled to allow for creation of an injectable slurry. For example, suitable freezer temperatures are less than about −20° C., between about −20° C. and about −15° C., −15° C. and about −10° C., between about −10° C. and about −5° C., between about −5° C. and about 0° C., or warmer than about 0° C. At the end of step 43, the biomaterial in the container may be in the form of an injectable ice slurry. In some embodiments, additional steps are required after step 43 to create an injectable ice slurry.

At step 44, the container is removed from the freezer and is either ready to be administered, i.e., is an injectable ice slurry, to a patient or subject. Alternatively, at step 44, the container is first subjected to the transformation methods previously described herein to ensure the container's contents are in the form of an injectable ice slurry. In addition to injection via a syringe, the slurry can be introduced into a patient at step 44 using any delivery system and/or technique known in the art. For example, if the solution (or slurry) is transported in a container (e.g., bag, vial), the solution can be transferred into an appropriate delivery device during any of steps 42-44 such as a cannula, a catheter, tubing, and/or a pump, and the like. A control device can control the flow rate, volume, and/or pressure of the injected slurry.

In an exemplary embodiment, the solution is prepared for shipment at step 40 in its aqueous phase at room temperature (i.e., without any ice content) by being placed in a sterile condition into a syringe, or sterilizing the solution after placement into the syringe. At step 41, the syringe with the solution placed inside of it is transported to the point of care, such as a medical facility (e.g., physician's office, clinic, hospital). Thus, the point of care receives a prefilled syringe. During step 41, the prefilled syringe is placed inside of a support vessel with transport protections such as bubble wrap or other padding. The solution that is initially prepared at room temperature maintains stability at step 41 while being transported at a variety of temperature conditions that may fluctuate throughout transport (e.g., due to natural weather conditions) including freezing temperatures below about 0° C., between about 0° C. and room temperature of about 20° C., and warm conditions of above 20° C. Transportation of the solution at a wide and fluctuating range of temperature conditions allows for simple and cost-effective transportation techniques well known in the art that do not require the use of special support vessels that maintain specific temperature ranges (e.g., freezing temperatures). However, in some embodiments, the solution is transported at sub-0° C. temperatures as discussed previously herein and will thus arrive at the point of care in a ready or near-ready state for administration to a subject or patient. A shipping vehicle (e.g., standard truck and/or airplane) is used for transport at step 41.

At step 42, the prefilled syringe is stored at the point of care. The syringe may either be stored inside the support vessel, or it may be removed from the support vessel upon arrival at the point of care. The prefilled syringe can be stored at room temperature for extended periods of time (e.g., several days to several months, and several months to one or more years), which allows for almost any point of care (e.g., physician's office, clinic, etc.) to stockpile the solution without requiring any specialized storage equipment or facilities. At step 43, the prefilled syringe is removed from storage and placed in a standard freezer that maintains a sub-0° C. temperature (e.g., at about −10° C.), allowing for the prefilled solution inside the syringe to at least partially crystallize. At step 44 the prefilled syringe is removed from the freezer and subjected to one or more methods of conditioning or transforming the ice contents into an injectable and flowable slurry (e.g., mechanical agitation, blending, mixing, vibration, ultrasonic energy, manual shaking, thawing) containing a percentage of ice particles (e.g., about 30% w/w) of particular sizes (e.g., about half of the internal diameter of the syringe needle), as previously described herein. The syringe contents are subjected to these state transformation methods without breaking the sterile barrier of the syringe. The support vessel in which the container is shipped may include the components necessary to transform the syringe contents into an injectable and flowable slurry. Finally, also at step 44 the slurry is injected into a patient at a target location for achieving prophylactic, aesthetic, and/or therapeutic results.

The injectable slurry described herein can be utilized to target all tissue types including, but not limited to, connective, epithelial, neural, joint, cardiac, adipose, hepatic, renal, vascular, cutaneous, and muscle tissues. The injectable slurry advantageously can focus a cooling effect directly at the site of the targeted tissue through injection directly into interstitial tissue, without the challenges of diffusion of heat or perfusion tissue, as described in the '042 application. As described in the '039 application, the injectable slurry can be used as a treatment for pain. Injection/infusion of the slurry near nerves causes crystallization of lipids in the myelin sheath, or direct cooling of non-myelinated nerves, thereby resulting in a site-specific relief of pain through inhibition of nerve conduction. The inhibition of peripheral nerve conduction is reversible (e.g., inhibition can occur for a period of minutes, days, weeks or months after a single administration of the slurry) (see the '039 application). In addition to pain relief applications, the injectable slurry can also be administered to target parts of the somatic and/or autonomic nervous system to treat a variety of conditions (e.g., inhibition of motor nerves to reduce muscle spasms, inhibition of sympathetic fibers that innervate the eccrine glands to reduce hyperhidrosis, inhibition of renal sympathetic nerves as a treatment for hypertension, and inhibition of neural input to the bladder to treat incontinence) (see the '039 application).

The devices, systems, and methods disclosed herein are not to be limited in scope to the specific embodiments described herein. Indeed, various modifications of the devices, systems, and methods in addition to those described will become apparent to those of skill in the art from the foregoing description and accompanying figures. Such modifications are intended to fall within the scope of the appended claims.

1. A method of transporting and preparing an injectable ice slurry for administration to a patient at a point of care, the method comprising:
preparing a biocompatible solution comprising water and a freezing point depressant;
placing the biocompatible solution into a syringe, wherein the syringe comprises a sealed internal volume;
transporting the syringe with the biocompatible solution to the point of care;
transforming the biocompatible solution into the injectable ice slurry within the syringe at the point of care; and
administering the injectable ice slurry from the syringe directly into a target tissue of the patient at the point of care,
wherein a sterile barrier of the syringe is not broken after placing the biocompatible solution into the syringe and prior to the administering.

2. The method of claim 1, wherein the freezing point depressant is glycerol.

3. The method of claim 1, wherein the biocompatible solution further comprises a salt.

4. The method of claim 3, wherein the salt is sodium chloride.

5. (canceled)

6. The method of claim 1, wherein an amount of the freezing point depressant is selected from the group consisting of about 30% (v/v) of the biocompatible solution, about 20% (v/v) of the biocompatible solution, and about 10% (v/v) of the biocompatible solution.

7. The method of claim 1, wherein the water constitutes about 80% (w/v) of the biocompatible solution.

8. The method of claim 1, wherein the biocompatible solution is configured to be transported to and stored at the point of care in a non-temperature-controlled environment prior to transforming the biocompatible solution into the injectable ice slurry.

9. The method of claim 1, wherein the modifying is selected from the group consisting of mechanical agitation, blending, mixing, vibration, ultrasonic energy, manual shaking, freezing, thawing, and a combination thereof.

10. The method of claim 1, wherein the syringe is transported to the point of care in a support vessel, and wherein the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of ice particles.

11. The method of claim 1, wherein the biocompatible solution is configured to be transformed into the injectable ice slurry by placing the syringe into a freezer at the point of care.

12. The method of claim 11, wherein the injectable ice slurry comprises a plurality of ice particles and is configured to flow through a lumen for administering the injectable ice slurry from the syringe directly into the target tissue of the patient.

13. A method of transporting a syringe that has been pre-filled with a biocompatible solution to a point of care, the method comprising:
preparing the biocompatible solution, wherein the biocompatible solution comprises water and a freezing point depressant;
placing the biocompatible solution into the syringe, wherein the syringe comprises a sealed internal volume; and
transporting the syringe with the biocompatible solution to the point of care in a support vessel,
wherein the biocompatible solution is transformed into an injectable ice slurry within the syringe at the point of care
wherein the syringe is configured to allow for direct administration of the injectable ice slurry from the syringe into a target tissue of a patient at the point of care, and
wherein a sterile barrier of the syringe is not broken after placing the biocompatible solution into the syringe and prior to the administration.

14. The method of claim 13, wherein the freezing point depressant is glycerol.

15. (canceled)

16. The method of claim 13, wherein the biocompatible solution is transformed into the injectable ice slurry by placing the syringe into a freezer at the point of care.

17. The method of claim 13, wherein the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of ice particles.

18. The method of claim 13, wherein the injectable ice slurry comprises about 10% ice by weight, between about 10% ice by weight and about 20% ice by weight, between about 20% ice by weight and about 30% ice by weight, between about 30% ice by weight and about 40% ice by weight, between about 40% ice by weight and about 60% ice by weight, or more than about 60% ice by weight.

19. A method of administering an injectable ice slurry at a point of care to a patient comprising:
receiving at the point of care a syringe comprising a biocompatible solution and a sealed internal volume;
transforming the biocompatible solution into the injectable ice slurry within the syringe at the point of care; and
administering the injectable ice slurry from the syringe directly into a target tissue of the patient at the point of care,
wherein a sterile barrier of the syringe is not broken after the receiving and prior to the administering, and
wherein the biocompatible solution comprises water and a freezing point depressant.

20. The method of claim 19, wherein the freezing point depressant is glycerol or a derivative thereof, and wherein the biocompatible solution further comprises sodium chloride or a derivative thereof.

21. The method of claim 19, wherein an amount of the freezing point depressant is selected from the group consisting of about 30% (v/v) of the biocompatible solution, about 20% (v/v) of the biocompatible solution, and about 10% (v/v) of the biocompatible solution.

22. The method of claim 19, wherein the syringe is received in a sealed state configured to maintain a sterility of the biocompatible solution and of the injectable ice slurry prior to the administration of the injectable ice slurry directly into the target tissue of the patient.

23. (canceled)

24. The method of claim 19, wherein the syringe is transported to the point of care in a support vessel, and wherein the support vessel is configured to transform the biocompatible solution into the injectable ice slurry after the biocompatible solution has been exposed to a temperature of between about −20° C. and about 0° C. for a period of time that is sufficient to at least partially transform the water in the biocompatible solution into a plurality of ice particles.

25. (canceled)

26. (canceled)

27. (canceled)

28. (canceled)

29. (canceled)

30. (canceled)

31. The method of claim 1, wherein the syringe comprises a visible temperature indicator configured to visually indicate a temperature of the biocompatible solution and to dynamically track the temperature over time.

32. The method of claim 31, wherein the visible temperature indicator is a label, a sticker, a marker, a crayon, a lacquer, or a pellet.

33. (canceled)

34. The method of claim 13, wherein the syringe comprises a visible temperature indicator configured to visually indicate a temperature of the biocompatible solution and to dynamically track the temperature over time.

35. (canceled)

36. The method of claim 34, wherein the visible temperature indicator is a label, a sticker, a marker, a crayon, a lacquer, or a pellet.

37. (canceled)

38. The method of claim 19, wherein the syringe comprises a visible temperature indicator configured to visually indicate a temperature of the biocompatible solution and to dynamically track the temperature over time.

39. (canceled)
